(19) United States
(12) Patent Application Publication
(10) Pub. No.: US 2003/0033023 A1
(43) Pub. Date: Feb. 13, 2003

(54) Arm mounted prosthetic device

(76) Inventor: William H. McCrae, Dallas, TX (US)

Correspondence Address: Daniel Rubin, The White House On Turtle Creek, 2401 Turtle Creek Blvd., Dallas, TX, 75219-4760, US

(21) Appl. No.: 09/925,152

(22) Filed: Aug. 8, 2001

(51) Int. Cl. A61F 2/54; A61F 2/56

(52) U.S. Cl. 623/65

(57) ABSTRACT

A prosthetic device for use by persons having a physically impaired hand and comprised of a rigid support to be securely mounted onto the forearm of the user. Permanently joined to the support is a metallic elongated tubular socket having a non-circular internal cross section to receive and secure a selected utensil terminating in a complementary cross section for an interfit within said socket.

### FIELD OF THE INVENTION

The field of art to which the invention relates comprises the art for an arm mounted prosthetic device for use by persons having a physically impaired hand.

### BACKGROUND OF THE INVENTION

Persons suffering from an impaired hand as having occurred congenitally, accidently or otherwise can be severely handicapped in their inability to perform every day tasks that a normal hand can provide. Whether holding a fork, screwdriver or other similar device, such persons are unable to secure the device and then perform the intended function that the device normally entails. Even though the related arm remains strong and supportive, the hand by itself cannot exert the thrust and turning motion, etc. necessary to perform the normal task associated with such devices. Eating accessories, for example such as a knife, fork, or spoon typically cannot be directed firmly enough to enable cutting up of a piece of meat or fish much less, closely approximating the ordinary use of such table implements in an unobtrusive tasteful manner.

### DESCRIPTION OF THE PRIOR ART

Various prosthetic devices for use by persons with an impaired hand are known to provide aid to somewhat overcome the handicap. Exemplifying such devices are the disclosure of U.S. Pat. Nos. 1,344,357; 3,434,163;3,802,302;3,942,194;4,944,766; and 5,597,189.

While each of the foregoing offer a contribution toward overcoming the handicap in issue, none are seen as affording the simplicity, versatility and unobtrusiveness of transmitting most or all of the thrusting or flexing of the forearm directly to the connected tool or device.

### OBJECTS OF THE INVENTION

An object of the invention is to provide novel apparatus for use by persons having an impaired hand that will enable the person to at least exercise a limited capability toward overcoming their handicap.

It is a further object of the invention to effect the previous object with a device able to deliver most or all of the thrusting strength of the person's forearm to a received tool or device.

It is a still further object of the invention to effect the foregoing objects in a reliable and economical manner.

### SUMMARY OF THE INVENTION

This invention relates to apparatus for use by persons having a physically impaired hand. More specifically, the invention relates to a device that can be readily mounted above the hand onto the underside of the forearm of the user so it can be operated by the forearm without any reliance on the hand per se. Typically it is worn within the shirt sleeve so as to be rendered inconspicuous.

For achieving the foregoing, the device hereof includes an elongated length of an arcuate section of thin metal plate of a composition such as stainless steel. A section of soft cushion material such as moleskin available from Johnson and Johnson is placed between the metal plate and the inner aspect of the arm to prevent shifting of the metal plate about the forearm and render the device more comfortable. The fabric can be readily wrapped tautly about the forearm and secured in place as a sleeve by use of hook and loop type fasteners (Velcro™), buttons, etc. Secured as by welding to the interior surface of the plate is an elongated length of a tubular socket shank internally defining a hexagonal or other suitable non-circular cross-section that would normally extend when installed to about halfway up the palm of the hand. The socket is adapted to releaseably receive and secure a utensil or utensil connection of complementary cross-section in a rotational interlock. The entire device can be worn beneath a shirt and/or jacket sleeve so as to be unobtrusive in a social setting such as a restaurant or the like.

A series of dining elements having the aforementioned non-circular ends can be attached to the socket shank of the arm sleeve as in a slender quick disconnect relation. Should the person using the device hereof have one good hand, the functioning hand can be readily used to add or change dining implements. Yet, all components other than the cloth straps and moleskin are made of dishwasher safe materials.

The device hereof can be advantageously utilized by virtually any adult or child with an impaired hand yet wise enough to be able to eat with knives and forks at a table and who has a desire to be able to do so relatively unassisted and unobtrusively. The foregoing can be effected in a manner compatible generally with what is loosely termed “good table manners” despite having a right or left hand and wrist disabled enough to preclude grasping a fork or spoon or knife firmly enough to deal with food (on a plate or food preparation surface) required to be sliced and cut and/or picked up in bite size morsels for introduction into the mouth.

In accordance herewith, the provided apparatus enables overcoming a long standing limitation imposed on persons having an impaired hand that cannot otherwise be utilized for the task contemplated.

The above noted features and advantages of the invention as well as other superior aspects thereof will be further appreciated by those skilled in the art upon reading the detailed description which follows in conjunction with the drawings.

### BRIEF DESCRIPTION OF THE DRAWINGS

FIG. 1 is a plan view of the prosthetic device hereof as positioned on the forearm underneath the shirt sleeve of the user;

FIG. 2 is interior view of the prosthetic device hereof in its open relation;

FIG. 3 is a fragmentary view of the underside of the device illustrating the socket shank secured to the metal plate;

FIGS. 4, 5, 6, 7 and 8 exemplify utensils that can be utilized and secured in the socket connection thereof;

FIG. 9 is a side view of the goblet holding utensil of FIG. 8; and

FIG. 9A is an enlarged view of the encircled portion of FIG. 9.

### DESCRIPTION OF THE PREFERRED EMBODIMENT

In the description which follows, like parts are marked throughout the specification and drawings with the same reference numerals respectively. The drawing figures are not necessarily to scale and in certain views, proportions may have been exaggerated for purpose of clarity.

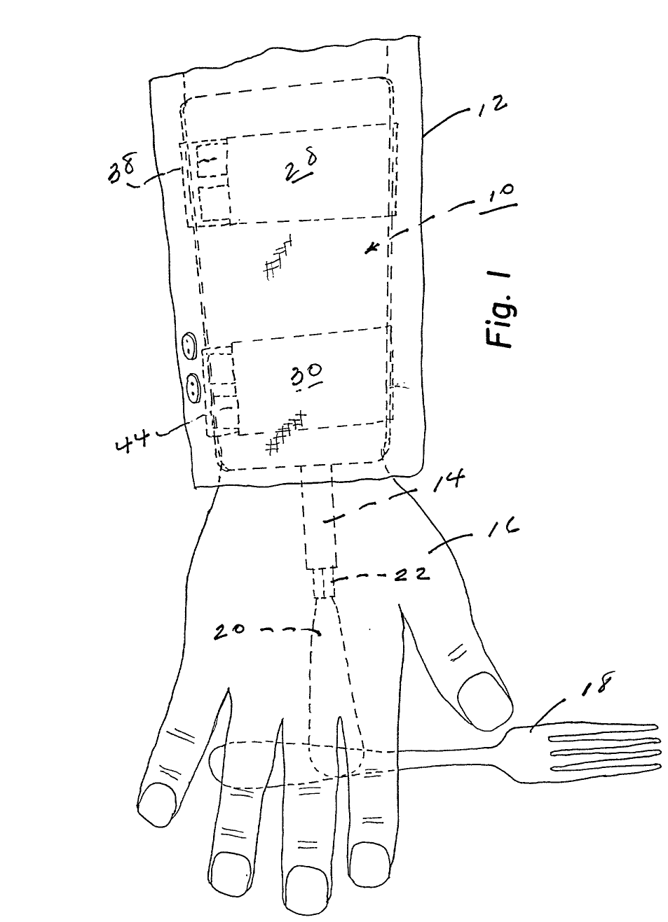

Referring now to FIG. 1, there is illustrated the arm mounted device hereof designated 10. The device is shown largely in phantom beneath a shirt sleeve 12 that includes an elongated tubular socket 14, to be described, extending to the palm underside of hand 16. Also shown in FIG. 1 is a utensil such as transverse fork 18 to be utilized and which includes a connector plate 20 that terminates in a bar 22 adapted to be received and secured in socket 14.

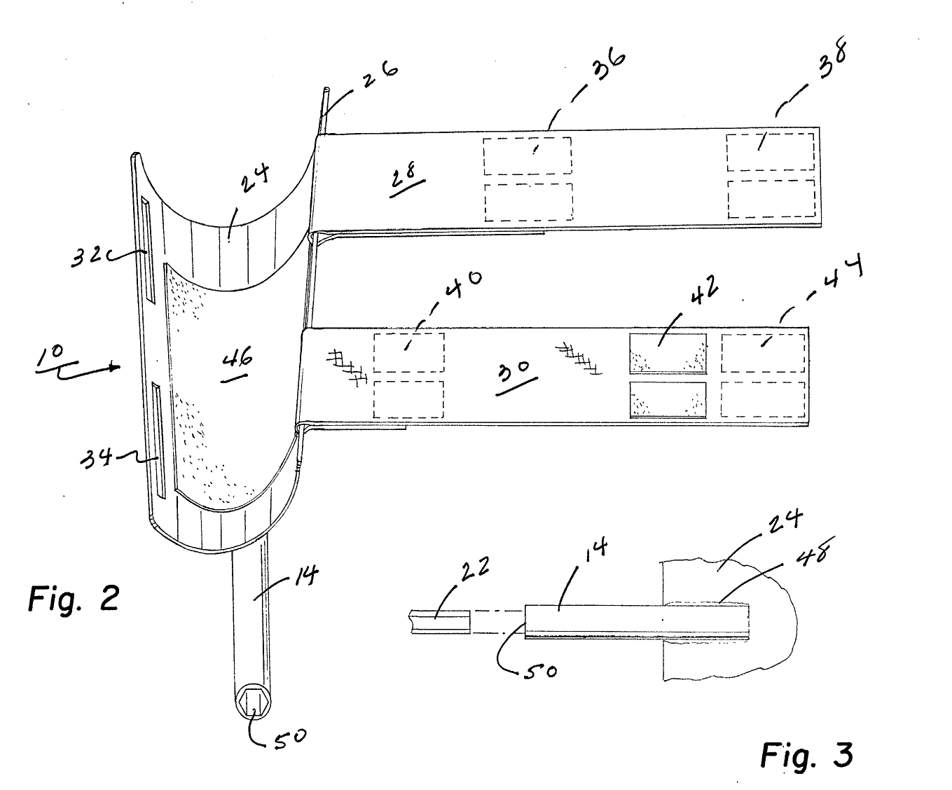

As best seen in FIGS. 2 and 3 hereof the device 10 is comprised of an arcuate section 24 of thin metal plate such as stainless steel that includes along one edge an elongated dowel 26 on which is mounted a pair of spaced apart fabric straps 28 and 30. Secured to each of the strap surfaces are a plurality of Velcro pairs 36, 38, 40, 42, and 44. Located through plate 24 along the opposite edge from dowel 26 is a pair of spaced apart slots 32 and 34 through which the straps 28 and 30 can extend for wrapping the device tautly onto the forearm of the user. By doubling back on the straps after passing through the slots engagement of the Velcro units is effected. A thin rectangular section of flexible liner padding 46 such as a woven cloth moleskin described supra is positioned on the interior face of metal plate 24 that serves to maintain positioning of plate 24 and minimize shifting if any. Moleskin is characterized as a woven cloth with a nap surface on one side and adhesive on the other side with the adhesive side placed against the metal plate.

Elongated tubular socket 14 is of a type commercially available and formed of a ridged hard metal composition that is secured to the exterior under surface of plate 24 as by a weld 48. The interior tubular portion 50 of socket 14 is of a non-circular section, such as hexagonal or the like, adapted to releaseably receive and secure a complementary end 22 of a utensil to be utilized in a rotational interlock therebetween. Obviously, the complementary sections of socket tube 50 and end piece 22 could be interchanged.

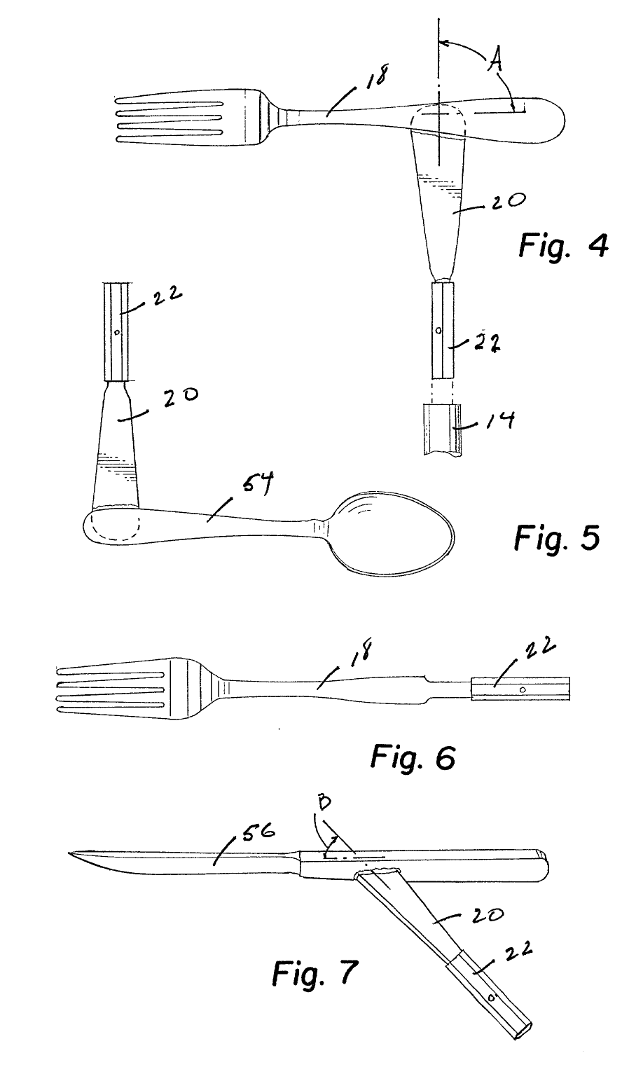

FIG. 4 illustrates a utensil in the form of fork 18 welded to a connector 20 having an end 22 of complementary cross section to be received and secured within tubular socket 14. In order to render the fork in an orientation most suitable for the user, connector 20 is joined to the fork 18 at an angle A that preferably is about ninety degrees.

FIG. 5 illustrates a spoon similarly joined while the fork of FIG. 6 lacks a connector 20 but instead extends rectilinearly to its distal end 22.

The knife of FIG. 7 includes a connector 20 having an end piece 22 secured to a knife handle at an offset angle B that preferably is about forty-five degrees.

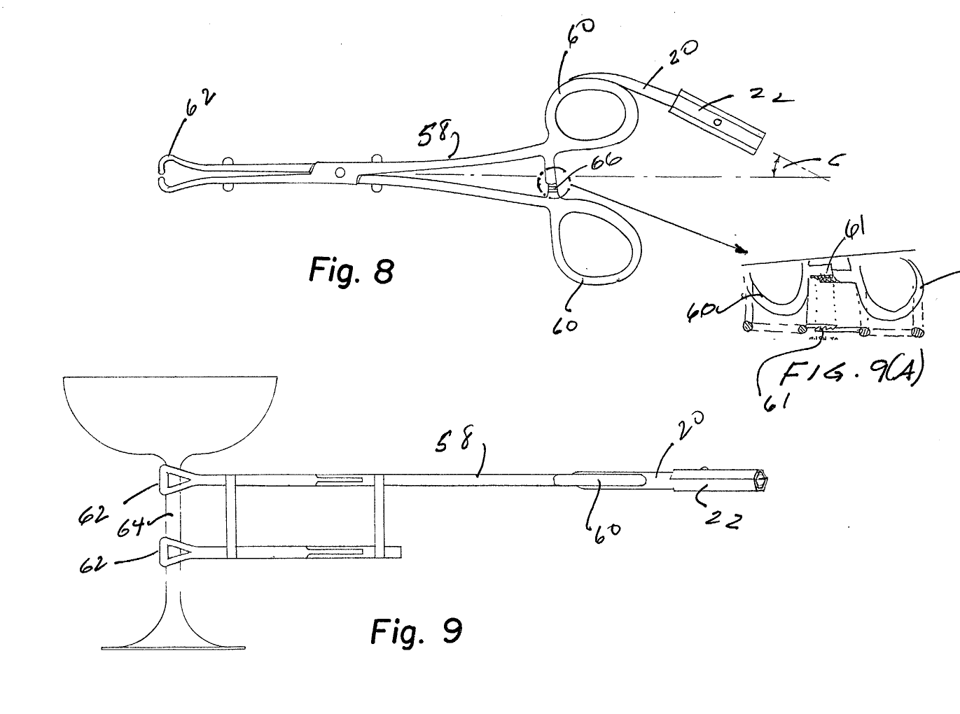

Finally, FIGS. 8 and 9 disclose a scissors type surgical clamp also known as a BABCOCK clamp, that includes a connector 20 and end piece 22 secured to one of the finger grips 60. The finger grips are drawn together with a simple compressive force and serve to maintain modifier grippers 62 in a closed relation about goblet stem 64 by means of ratchet lock 66. (FIG. 9A) Releasing stem grippers 62 is effected by twisted or pushing lock 66 at a slant until causing the ratchet lock to disengage. Preferably, the grasping jaws 62 are altered so that the grasp force around a comparatively fragile glass stem of the drinking goblet is delivered over a long enough area of contact to dissipate the compressive force sufficiently to render fracture of the glass stem at that point unlikely even when the vessel is fairly full of beverage. The optimum length of jaws contact for this purpose has been found to be about 2 centimeters.

By the above description there is disclosed a novel prosthetic device for use by a hand impaired individual. The device is easily mounted on the forearm with plate 24 extending along the underside of the arm and straps 28 and 30 inserted and secured so as to maintain the device in a taut wrapped relation. A utensil modified to include an end piece for intermitting within the socket 14 enables the user to employ the thrust and force of his forearm to perform the contemplated function with the utensil.

The entire weight of the holder device is only a few ounces, yet it can transmit, via the forearm, several pounds of thrust, lateral or rotational movement as required. The entire device except for a forward portion of the socket is maintained concealed in the palm of the hand yet lacks any mechanical relationship to the hand whether the latter has residual function or not. The arm piece and socket allow several arm forces to be transmitted through the device and to any attachment including a longitudinal thrust as with a straight-ahead alignment of a modified dinner fork or perhaps a screwdriver or garden weeding device. Transverse or “flex-extended” movements of the forearm and arm can be readily achieved for scooping up food for transport to the mouth or, for example, in sandpapering a wooden surface.

Moreover, the device is structured so as to significantly conceal its existence on the user. That is, being contained largely within the shirt sleeve, its presence is not readily noticeable and thus avoids drawing attention to the diner's disability that many people consider unpleasant. Unlike previous devices therefor, it affords the user more nearly normal dinner eating movements that are less conspicuous to his or her disability whether at a stand up buffet meal or at a seated dinner.

Yet again, the device and all dining implements can be washed virtually hospital clean by being placed in an ordinary dishwasher after removing the web strapping that is clothes-washer durable. Every component other than the web strapping is stainless steel and thus readily washable by one or the other. The moleskin lining 46 of the steel arm sleeve can be replaced from time to time by ordinary off-the-shelf shoe-liner pads obtained from a pharmacy. A drop or puff of a light lubricating oil or spray is recommended for the inside of socket 14 after washing in ordinary soapy water. Manufacture of the device requires no special spring steels or other specialty stock items, only flat stainless stock and commercially available hexagonal bit sockets for receiving and holding any utensil selected for use.

Although the emphasis above is primarily on use with dining and modified dining implements, it is to be understood that the device can be opportunely utilized to attach a wide variety of small hand tools, grasping clamps, paint brushes and the like, only requiring modification sufficient to allow attachment of the hexagonal male component to interfit with the receiving socket on the arm device.

Since many changes could be made in the above construction and many widely different embodiments of this invention could be made without departing from the scope thereof, it is intended that all matter contained in the drawings and specification shall be interpreted as illustrative and not in a limiting sense.

1. A prosthetic device for use by a person having a physically impaired hand, the device comprising:
a) a support adapted to be wrapped mounted onto the forearm of the person's hand that is impaired;
b) an elongated tubular socket rigidly secured to said support for receiving and securing a utensil releaseably installed in a rotational interlock therewith; and
c) a selection of different utensils useful individually for cooperative installation with said socket so as to extend beyond said socket into the vicinity of said hand, said utensils when installed being responsive to forces exerted by said person's forearm to simulate the contemplated use of the selected utensil.

2. A prosthetic device in accordance with claim 1 in which said support includes a thin arcuate metal section adapted to engage the underside of the person's forearm when installed.

3. A prosthetic device in accordance in claim 2 in which said socket is rigidly secured to said metal plate and extends in the direction of said forearm.

4. A prosthetic device in accordance with claim 3 in which said socket is of metal composition and is welded to said metal plate to be rigidly secured thereto.

5. A prosthetic device in accordance with claim 4 including a liner fabric of layered composition on said support intervening between said metal plate and the person's forearm on which the metal plate is to be installed so as to urge retention against displacement of said metal plate in its installed relation against the forearm.

6. A prosthetic device in accordance with claim 5 in which said socket is internally tubular of non-circular cross section and each of said utensils is of metal including an end of complementary cross section to said socket to releaseably interfit therein.

7. A prosthetic device in accordance with claim 6 in which the non-circular section of said socket is hexagonal.

8. A prosthetic device in accordance with claim 1 in which at least some of the utensils include a metal connector joined intervening between the utensil and their interfit end for offsetting the directional orientation of said utensil for said contemplated use.

9. A prosthetic device in accordance with claim 8 in which said different utensils are selected from a group consisting of a fork, a spoon, a knife and a goblet holder.

10. A utensil useful individually for cooperative installation with a socket of a prosthetic device on the arm of a user so as to extend beyond said socket into the vicinity of the user's hand, said utensil when installed being responsive to forces exerted by said person's forearm through said prosthetic device to effect the contemplated use of the utensil.

11. A utensil in accordance with claim 10 in which said socket is internally tubular of non-circular cross section and of a metal composition including an end of complementary cross section to said socket to releaseably interfit therein.

12. A utensil in accordance with claim 11 in which said utensil includes a metal connector joined intervening between the utensil and their interfit end for offsetting the directional orientation of said utensil for said contemplated use.

13. A utensil in accordance with claim 10 in which different utensil can be selected from a group consisting of a fork, a spoon, a knife and a goblet holder.
